(19) United States
(12) Patent Application Publication
(10) Pub. No.: US 2026/0060849 A1
(43) Pub. Date: Mar. 5, 2026

(54) Special Needs Diaper indicator (Global Braille/Sign Language)

(71) Applicant: Vanessa Bracetty, Brooklyn, NY (US)

(72) Inventor: Vanessa Bracetty, Brooklyn, NY (US)

(73) Assignee: Vanessa Bracetty, Brooklyn, NY (US)

(21) Appl. No.: 18/742,569

(22) Filed: Aug. 30, 2024

(51) Int. Cl. A61F 13/42; A61F 13/49; A61F 13/84

(52) U.S. Cl. CPC A61F 13/42; A61F 13/49; A61F 2013/422; A61F 2013/424; A61F 2013/49098; A61F 2013/8479

(57) ABSTRACT

The present invention relates to a diaper indicator specifically designed for individuals with special needs, including those with visual and hearing impairments. The diaper indicator features embedded sensors that detect moisture and soiling within the diaper, providing accessible notifications through multiple channels. These channels include a Braille Tactile Code output panel for tactile notifications, a visual display that shows sign language images or animations. The invention is designed to be made from hypoallergenic materials to ensure user safety and comfort. This innovative diaper indicator enhances accessibility and ease of care for special needs individuals and their caregivers.

### BACKGROUND OF THE INVENTION

### (1) Field of the Invention

The present invention relates to the field of incontinence, specifically to diaper indicators designed for individuals with special needs, such as those with visual and hearing impairments. The invention integrates advanced accessibility features, including Braille Tactile Code notifications and sign language visual displays, to provide inclusive and effective diaper status alerts for users with disabilities. It addresses the challenges faced by caregivers and healthcare providers in managing the incontinence needs of individuals with sensory impairments, offering a comprehensive solution that enhances care and communication.

### BRIEF SUMMARY OF THE INVENTION

Creating a detailed specification for a diaper indicator that incorporates Braille Tactile Code and Sign Language (ASL) images involves focusing on accessibility features that cater to users with visual or hearing impairments, in addition to the standard functionalities of a diaper indicator. Below is a comprehensive outline:

### 1. Objective

To develop a diaper indicator that provides accessible notifications through Braille and sign language images/tactile code, ensuring that individuals with visual or hearing impairments, as well as their caregivers, can effectively monitor diaper status avoiding health issues.

### 2. Target Audience

Primary Users: All-inclusive with special needs individuals, particularly those with visual impairments (blind or low vision) and hearing impairments (deaf or hard of hearing), as well as infants and elderly individuals.
Secondary Users: Caregivers, parents, nurses, and healthcare professionals who may also have visual or hearing impairments.

### 3. Functional Requirements

### 3.1 Sensing Mechanism

Moisture Detection:
   Type: Capacitive, resistive, or humidity-based sensors embedded in the diaper
   Sensitivity: Capable of detecting varying levels of moisture to distinguish between minor dampness and excessive wetting.
   Accuracy: High accuracy to ensure reliable notifications without false alerts.
Soiling Detection:
   Optional Sensor: Chemical or pH sensor to detect fecal matter
   Sensitivity: Designed to detect specific markers of stool for accurate identification.

### 3.2Alert System

Visual Alert with Braille Integration:
   Display: Color changing (yellow to blue and/or safest color placement) integrated with a Tactile code Braille output.
   Braille Panel: A responsive Braille tactile code vertically placed in the front middle portion of the diaper that raises colored Braille dots to indicate the diaper status.
      Wet indicator: Braille indicator reading out “WET”
      Soiled Indicator: Braille indicator reading out “SOILED”
      Full Indicator: Braille indicator reading out “FULL”.
Visibility: Color changing should be bright enough for visibility with a distance
Sign Language Images (ASL):
   Display: Color changing image vertically placed in the front and/or back portion of the Diaper aligned in the middle that shows sign language images or animations indicating the diaper's status.
      Wet Indicator: An image or animation of the American Sign Language (ASL) sign for “Full and/or “WET”
      Soiled Indicator: An image or animation of the ASL sign for “DIRTY”, “POOP” and/or “SOILED”

### 3.3 Safety Features

Skin Safety: Use hypoallergenic, non-toxic, and biocompatible materials to prevent irritation.

### 3.4 Usability

Ease of Attachment: Simple mechanism to attach to various diaper types (Velcro, adhesive), accessible for users with visual impairments.
Size and weight: Compact, lightweight design to avoid discomfort and allow ease of use.
Durability: Strong enough to withstand daily use, resistant to drops and normal wear.

### 4. Testing and Validation

Prototype Testing: Involving visually and hearing-impaired individuals and their caregivers to gather feedback and refine the design
Certification: Obtaining necessary certifications and approvals from relevant health and safety authorities.

### 5. Ethical Considerations

Inclusivity: The product should be designed to be fully inclusive, respecting the dignity and privacy of all users.
Sustainability: Commitment to using environmentally friendly materials and processes, minimizing the environmental impact.

### BRIEF DESCRIPTION OF THE SEVERAL VIEWS OF THE DRAWING(S)

FIG. 1 shows a semi full front view of the diaper with braille tactile code and sign language images displayed on the front of the diaper

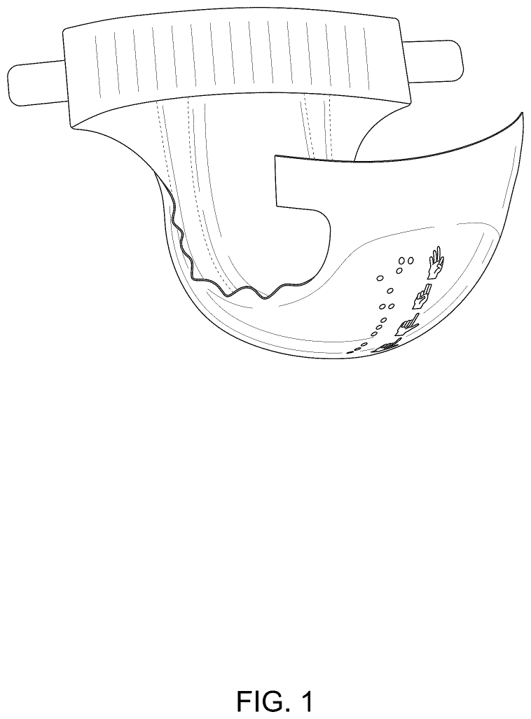

FIG. 2 shows the full front diaper view with braille tactile code and sign language images of the word “FULL” in a vertical form. The words may read “FULL”, “WET”, or “SOILED” and will always be in a vertical form

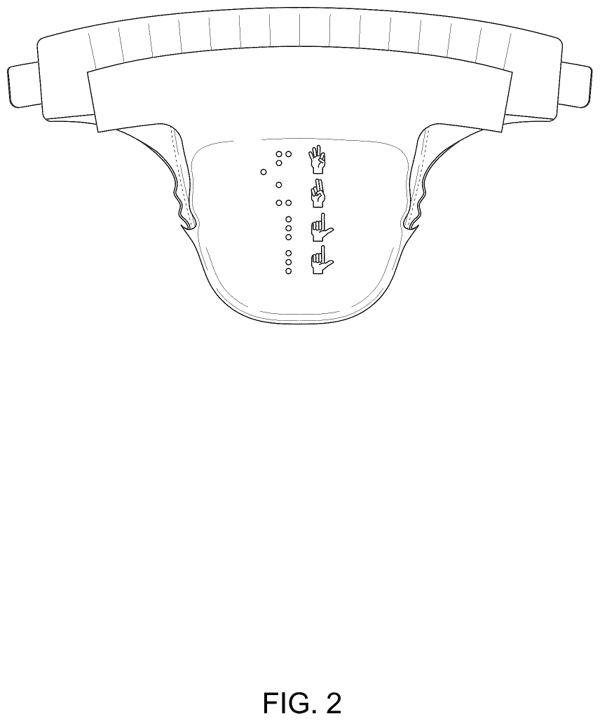

FIG. 3 shows the back view with braille tactile code and sign language images of the word “FULL” in a vertical form. The words may read “FULL”, “WET”, or “SOILED” and will always be in a vertical form

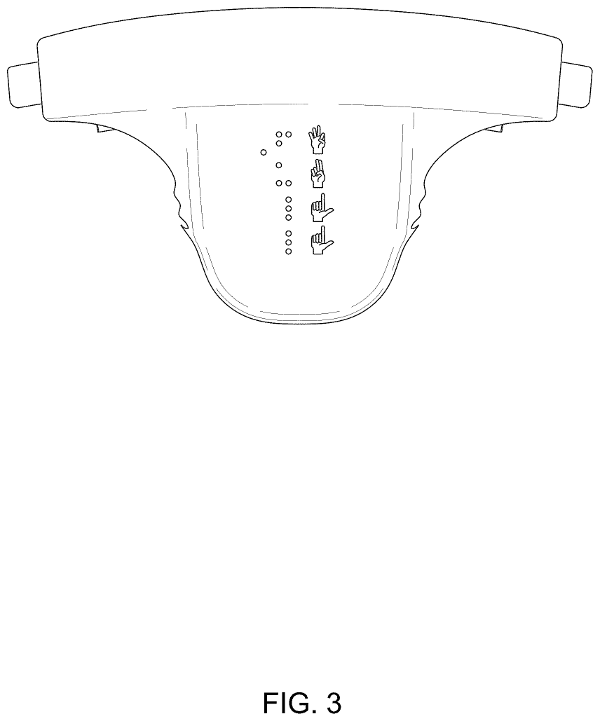

FIG. 4 shows the inside of the diaper

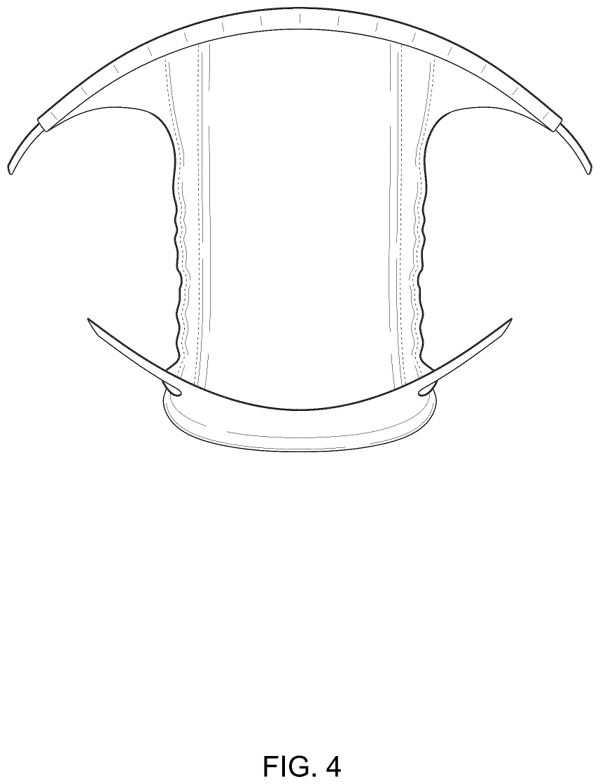

FIG. 5 shows the bottom of the diaper with front and back views of braille tactile code and image indicator as it would be placed either in the front of the diaper and/or in the front and back of the diaper.

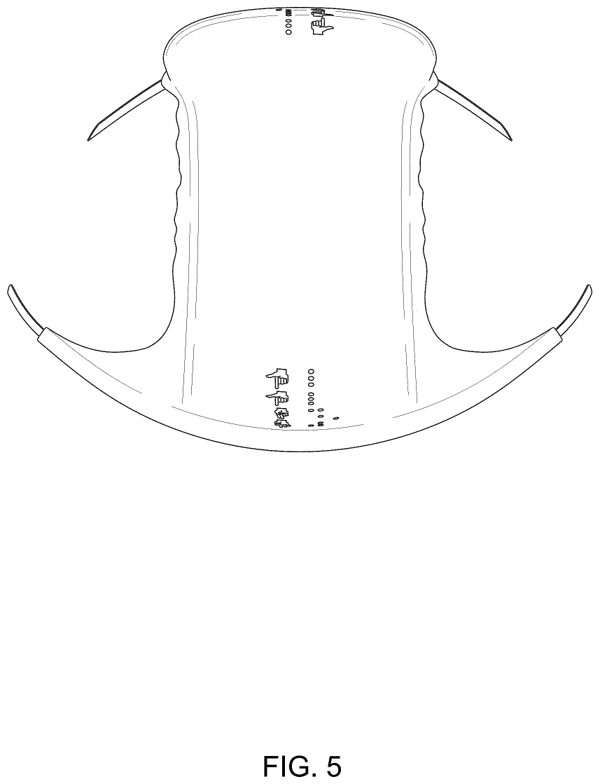

FIG. 6 Shows the Right Side of the Diaper

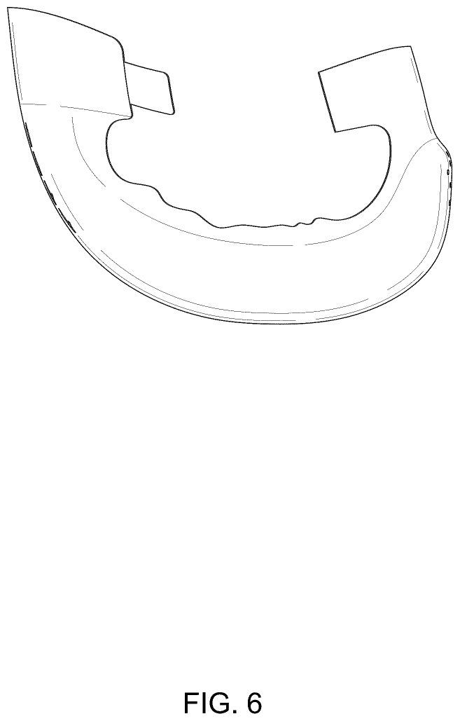

FIG. 7 shows the left side of the diaper

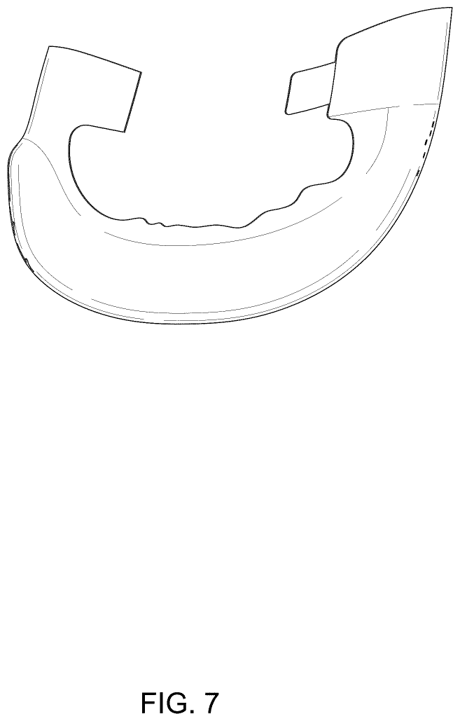

### DETAILED DESCRIPTION OF THE INVENTION

The invention is an all-inclusive specialized diaper with an indicator designed for individuals with special needs, particularly those with visual or hearing impairments. It features integrated sensors that detect fullness, moisture and soiling within the diaper, providing timely alerts through accessible channels.

Key innovations include a Braille panel that displays tactile notifications for visually impaired users, such as “WET”, “FULL” or “SOILED”, and a display that shows sign language images or animations, indicating the diaper's status for those who are deaf or hard of hearing.

This diaper indicator is designed to be user-friendly making it a reliable and inclusive solution for managing incontinence care in special needs populations.

1. A diaper indicator for special needs individuals, comprising:
A color visual indicator within the diaper to detect moisture and/or soiling
A Braille Tactile code output panel integrated into the indicator, configured to display tactile code notifications in Braille, indicating the status of the diaper (e.g., “WET”, “SOILED”, or “FULL”)
A visual display capable of showing sign language images or animations corresponding to the diaper's condition.
The diaper indicator of claim 1, wherein the Braille Tactile code output panel is responsive and dynamically informs based on real-time exposure to wetness, providing immediate tactile feedback.
The diaper indicator of claim 1, wherein the visual display supports multiple sign Languages and provides clear, high-contrast images or animations for users who are Deaf or hard of hearing.
The diaper indicator of claim 1, wherein the indicator is designed and made of hypoallergenic materials to ensure safety and comfort for the user.
